(19) United States
(12) Patent Application Publication
(10) Pub. No.: US 2009/0222017 A1
(43) Pub. Date: Sep. 3, 2009

(54) POSITIONING BRACKET

(76) Inventor: Robert Lye, New South Wales (AU)

Correspondence Address: HENRICKS SLAVIN AND HOLMES LLP;SUITE 200, 840 APOLLO STREET, EL SEGUNDO, CA, 90245, US

(21) Appl. No.: 12/162,744

(22) PCT Filed: Jan. 31, 2006
(86) PCT No.: PCT/AU06/00111

§ 371 Date: Dec. 2, 2008

(51) Int. Cl. A61B 17/90

(52) U.S. Cl. 606/91

(57) ABSTRACT

A positioning bracket for a surgical procedure to assist in determining the correct placement and/or alignment of a surgical device for a patient, said positioning bracket comprising a first arm (1) mounted on a support frame (6,7), a rear positioning pad (4) located on the first arm (1) adjacent an end of the first arm for placement on the patient, a front positioning pad (2) located on the first arm (1) adjacent the other end of the first arm for placement on the patient, and a second arm (8) mounted on the support frame (6,7), the second arm (8) comprising a connector (12) adjacent an end of the second arm (8) to receive a surgical device, wherein the support frame (6,7) permits the connector (12) to be positioned relative to the rear positioning pad (4) and front positioning pad (2).

### FIELD OF THE INVENTION

The present invention relates to surgical implements and surgical methods and in particular to a positioning bracket for use in surgical procedures, for example surgery involving prosthetic components.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Whilst the following discussion is with respect to hip replacement surgery, a person skilled in the art will appreciate that the present invention is not limited to this particular field of use and may be adapted for use with other types of surgery.

Hip replacement surgery involves the use of a prosthetic cup (acetabular cup) and a prosthetic ball (femoral head) to restore the ball and cup joint functionality of the hip. The ball and cup joint enables the hip to rotate in different directions to various degrees (in contrast to the relatively limited rotation of a knee joint).

In 2001, approximately 165,000 total hip replacements were performed, according to data from the American Academy of Orthopaedic Surgeons, using figures from the National Center for Health Statistics. Historically, hip replacement (arthroplasty) surgery required up to a 40 cm (7 to 12 inches) curved incision to provide sufficient access for the surgeon to manually access and manipulate the hip and femur. A prosthetic cup was attached to the hip socket and the head of the femur removed and replaced with a prosthetic ball.

After the incision is made, the ligaments and muscles are separated to allow the surgeon access to the bones of the hip joint. It is this part of the surgery that makes the ligaments and muscles somewhat weak after surgery. Until they heal, which takes about a month to six weeks, the patient must follow special hip precautions to prevent dislocation of the new hip joint.

Removing the Femoral Head: Once the hip joint is entered, the femoral head is dislocated from the acetabulum. Then the femoral head is removed by cutting through the femoral neck with a power saw.

Reaming the Acetabulum: After the femoral head is removed, the cartilage is removed from the acetabulum using a power drill and a special reamer. The reamer forms the bone in a hemispherical shape to exactly fit the metal shell of the acetabular component.

Inserting the Acetabular Component: A trial component, which is an exact duplicate of the patient's hip prosthesis, is used to ensure that the joint received will be the right size and fit. Once the right size and shape is determined for the acetabulum, the acetabular component is inserted into place. In the uncemented variety of artificial hip replacement, the metal shell is simply held in place by the tightness of the fit or with screws to hold the metal shell in place. In the cemented variety, a special epoxy type cement is used to “glue” the acetabular component to the bone.

Preparing the Femoral Canal: To begin replacing the femoral head, special rasps are used to shape and hollow out the femur to the exact shape of the metal stem of the femoral component. Once again, a trial component is used to ensure the correct size and shape. The surgeon will also test the movement of the hip joint.

Inserting the Femoral Stem: Once the size and shape of the canal exactly fit the femoral component, the stem is inserted into the femoral canal. Again, in the uncemented variety of femoral component the stem is held in place by the tightness of the fit into the bone (similar to the friction that holds a nail driven into a hole drilled into wooden board—with a slightly smaller diameter than the nail). In the cemented variety, the femoral canal is rasped to a size slightly larger than the femoral stem. Then the epoxy type cement is used to bond the metal stem to the bone.

Attaching the Femoral Head: The metal ball that replaces the femoral head is attached to the femoral stem.

The Completed Hip Replacement: Before the incision is closed, an x-ray is taken to make sure the new prosthesis is in the correct position.

Such surgery had a number of problems including:
a hospital stay of three days or more, post-operative pain and weeks of rehabilitation;
each centimetre of incision has a tenfold increase in the risks of blood clotting and infection post surgery; and
the surgeon was reliant on his experience and eye to ensure accurate placement and of the cup into the three dimensional hip socket and alignment of the cup with the ball/femur to enable proper function of the joint. Misalignment may lead to post operative complications such as misalignment of the leg, incorrect leg length and/or incorrect soft tissue tension. The long term effects of misaligned prosthetic components can also include accelerated wear of the components, aseptic loosening of the components and potentially early repetition of the surgery.

Attempts to overcome these problems include:
WO 2003/037192 which discloses a jig (impaction tool) for use in bone surgery and thus enables the use of a smaller incision. For hip replacement surgery, the jig enables the use of a 4 to 7 cm (2 to 3 inches) incision, ie keyhole surgery. Other benefits include a shorter stay in hospital, less blood loss, less pain, fewer postoperative dislocations and faster recovery; and
WO 2005/046475 which discloses a gauge to assist the surgeon with accurate placement of a prosthetic when using a jig in keyhole surgery as the surgeon is no longer able to see the fit of the cup into the hip socket or the fit between the ball and cup.

The gauge provided in WO 2005/046475 has enabled efficient use of the jig (impaction tool) of WO 2003/037192. Commercial examples include the NilNav Hip System available from MAC surgical. However, the gauge only works in two dimensions and requires the patient to be braced into a specific position since the angles measured by the gauge are specific for a precise position of the patient's hip. There is also still a heavy reliance on the surgeon's eye and experience for optimal placement of the cup into the hip.

There is thus a need for further aids to assist the surgeon during surgery, especially for surgeons who do not have significant experience in a particular surgical procedure.

### SUMMARY OF THE INVENTION

According to a first aspect of the invention, there is provided a positioning bracket for a surgical procedure to assist in determining the correct placement and/or alignment of a surgical device for a patient, said positioning bracket comprising:
(a) a first arm mounted on a support frame;
(b) a rear positioning pad located on the first arm adjacent an end of the first arm for placement on the patient;
(c) a front positioning pad located on the first arm adjacent the other end of the first arm for placement on the patient; and
(d) a second arm mounted on the support frame, the second arm comprising a connector adjacent an end of the second arm to receive a surgical device;
wherein the support frame permits the connector to be positioned relative to the rear positioning pad and front positioning pads.

The front and rear positioning pads are designed to be attached to specific parts of the body. This specific positioning results in the surgical device being appropriately positioned for accurate positioning and alignment for the surgical procedure. The invention thus provides the advantage of reducing the amount of subjective judgment that a surgeon needs to exercise during surgery. The invention is thus particularly useful in places where a particular type of surgery is not conducted often by the resident surgeons.

For example, in hip surgery, the rear positioning pad would be positioned over the patient's sacrum, one front positioning pad would be positioned over the patient's public tubercle and two other front positioning pads would press against the anterior superior iliac spine. The positioning bracket thus enables accurate positioning and alignment of the cup impactor.

In a preferred embodiment, either or both the positioning pads is/are movable and/or adjustable relative to the first arm.

In a preferred embodiment, the first arm is movable relative to the support frame. Preferably, the first arm is movable along the support frame. In a further preferred embodiment, the positioning bracket further comprises a lock to releasably fix the position of the first arm on the support frame.

In a preferred embodiment, the second arm is movable relative to the support frame. Preferably, the second arm is movable along the support frame. In a further preferred embodiment, the positioning bracket further comprises a lock to releasably fix the position of the second arm on the support frame.

In a preferred embodiment, the positioning bracket further comprises connector is movable relative to the second arm. Preferably, one end of the connector is slidingly engaged in an opening in the second arm. In a further preferred embodiment, one end of the connector includes an opening to receive the surgical device.

In a preferred embodiment, the support frame comprises a first support member and a second support member. Preferably, the first arm is mounted on the first support member. More preferably, the first arm is movable relative to the first support member. Most preferably, the first arm is movable along the first support member. In a further preferred embodiment, the positioning bracket further comprises a lock to releasably fix the position of the first arm on the first support member.

In a preferred embodiment, the second arm is mounted on the second support member. Preferably, the second arm is movable relative to the second support member. More preferably, the second arm is movable along the second support member. In a further preferred embodiment, the positioning bracket further comprises a lock to releasably fix the position of the second arm on the second support member.

In a preferred embodiment, the first support member and the second support member are movable relative to each other. Preferably, the first support member and the second support member are slidingly connected to each other. More preferably, the positioning bracket further comprises a lock to releasably fix the position of the first support member relative to the second support member.

According to a second aspect of the invention, there is provided a pelvic bracket for hip surgery to assist in determining the correct placement and/or alignment of an acetabulum cup for a patient, said pelvic bracket comprising:
(a) a first arm mounted on a support frame;
(b) a rear positioning pad located on the first arm adjacent an end of the first arm for placement on the sacrum of the patient;
(c) a front positioning pad located on the first arm adjacent the other end of the first arm and comprising three location sites, two of which are for placement on the patient's anterior superior iliac spine and the third is for placement on the patient's pubic tubercle; and
(d) a second arm mounted on the support frame, the second arm comprising a connector adjacent an end of the second arm to receive an acetabulum cup impactor;
wherein the support frame permits the connector to be positioned relative to the rear positioning pad and front positioning pads.

Further, the positioning bracket is adjustable to be fitted to either the left or right side of a patient. Some of the bracket parts may need to be changed for left or right side procedures.

### BRIEF DESCRIPTION OF THE DRAWINGS

Various embodiments/aspects of the invention will now be described with reference to the following drawings in which,

FIG. 1 is a drawing of a first embodiment of a positioning bracket according to the invention;

FIG. 2 is a drawing of the detail of a surgical device attached to the bracket in FIG. 1.

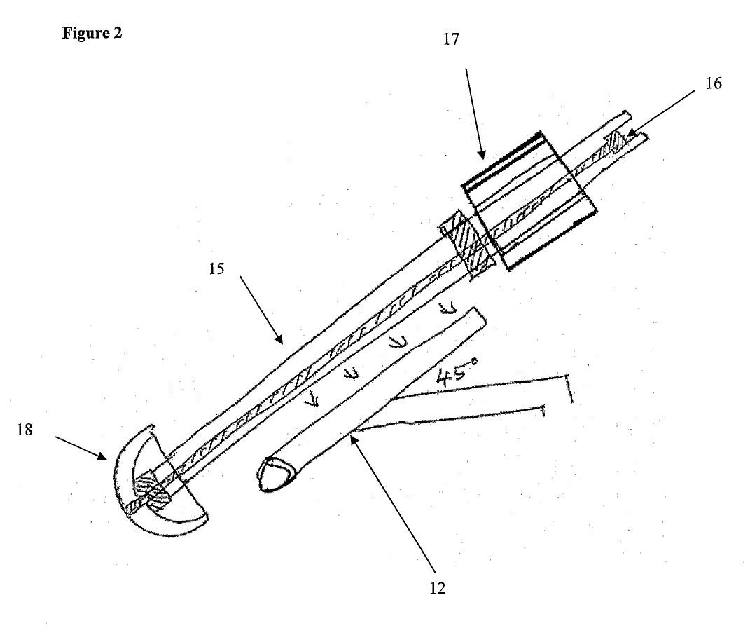

FIG. 3 is a drawing of the detail of the adjustable locks in a second embodiment of the invention.

### DETAILED DESCRIPTION OF A PREFERRED EMBODIMENT

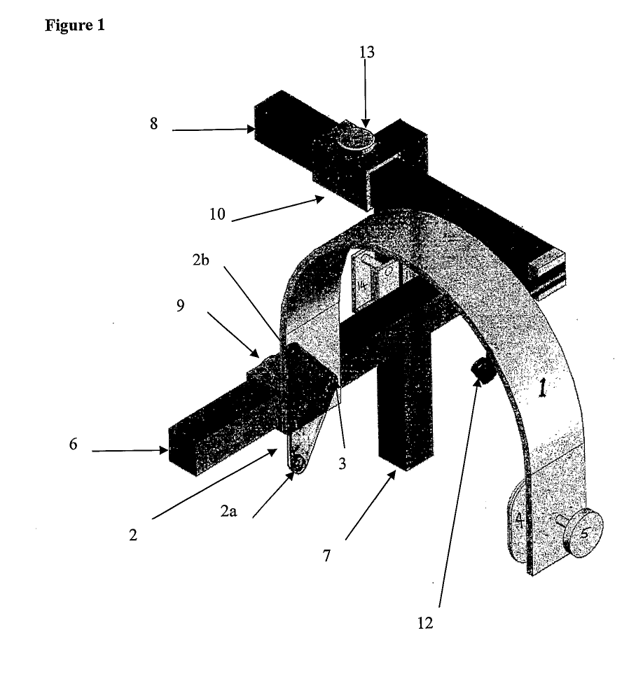

FIG. 1 illustrates a positioning bracket for use in a hip surgery. A first arm (1) is mounted on a support frame consisting of support members (6,7). Rear positioning pad (4) is located adjacent one end of the first arm (1). Front positioning pad (2) is located adjacent the other end of first arm (1). Second arm (8) is mounted on the support frame and comprises connector (12) adjacent one end to receive a surgical device, in this case an acetabulum cup impactor (15).

The first arm (1) is movable relative to the support frame and is movable along a first support member (6). Lock (9) releasably fixes the position of the first arm (1) on support member (6). Rear positioning pad (4) and front positioning pad (2) are both movable/adjustable relative to the first arm to enable use on left and right side hips.

The second arm (8) is movable relative to the support frame and is movable along a second support member (7). Lock (10) releasably fixes the position of the second arm (8) on support member (7).

The two support members (6,7) are movable relative to each other and are slidingly connected to each other. Lock (11) releasably fixes the position of the first support member (6) on second support member (7)

The patient (not shown) is positioned lying along the bed, parallel to the edge of the bed, on its side with the pelvis vertical to the bed. The first arm (1) is lowered over the patient and the front positioning pad (2) having 3 location sites (2a, 2b, 3) is aligned with the front of the patient. Location site (3) is aligned with the Pubic Tubercle. Location sites (2a, 2b) are aligned with the Anterior Superior Iliac Spine and must be in a vertical plane to the bed/patient. This can be achieved by eye, feel or by using a swivel gravity pin hinged in bracket (14) to indicate vertical. Rear positioning pad (4) is then tightened against the Sacrum with a lockable adjuster (screw) (5).

The locks (clamps) (9, 10, 11) are loosened and the support members (6, 7) and second arm (8) are adjusted to position the connector (12) in line with the reamed acetabulum. The locks (9, 10, 11) are now tightened to make the connector (12) rigid to the frame/patient. The connector is set at 45° and 15° aversion to the patient for correct positioning of the cup (18) into the acetabulum.

The cup impactor (15) is now inserted through connector (12). The acetabulum cup (18) is then attached to the cup impactor (15) via long screw (16). The cup (18) is now tightened onto the cup impactor (15) by tightening the screw head (16). The cup impactor (15) is now moved down through connector (12) until the cup (18) is in position in the reamed acetabulum. The support members (6, 7) and second arm (8) may have to be adjusted again to achieve this. When in the correct position, cup (18) is impacted into the acetabulum by the slap hammer (17). The screw (16) is now unscrewed from the cup (18) and whole procedure is now reversed to remove the positioning bracket from the patient.

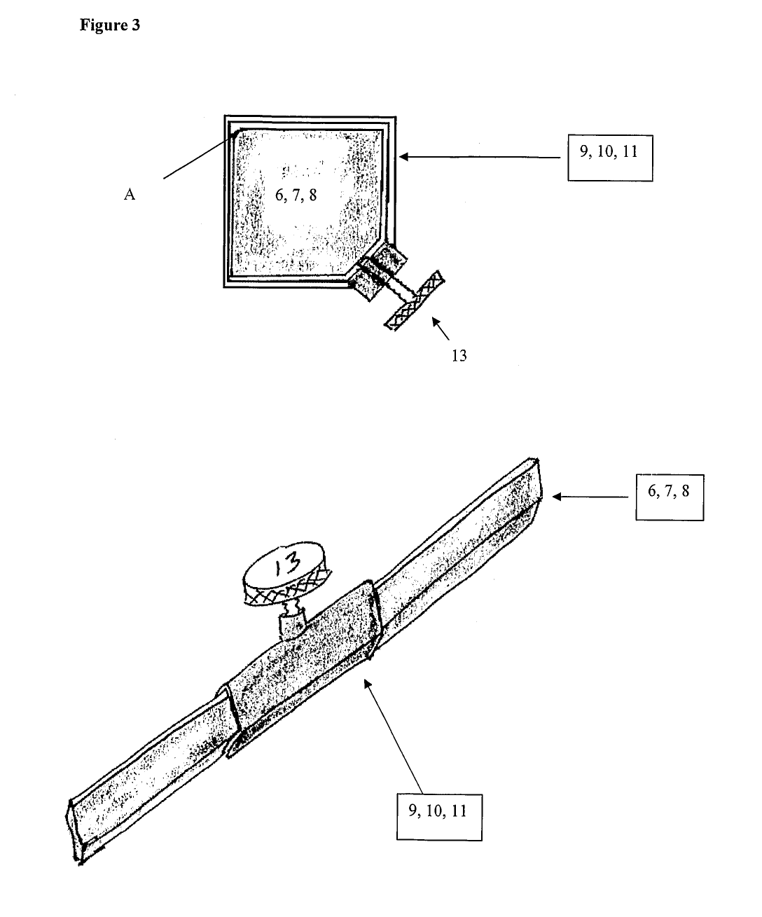

The detail of an alternative fit between the support members (6, 7) and second arm (8) and locks (9, 10, 11) is shown in FIG. 3. The support members (6, 7) and second arm (8) are rotated 90° so that the locking screws (13) act upon a corner of the support members (6, 7) and second arm (8). To provide for a better locking action, the corner of the support members (6, 7) and second arm (8) which interacts with the screw (13) is flattened to provide a surface for the screw to act against. In addition, the opposite corner (A) of support members (6, 7) and second arm (8) is also flattened to enable the arm to seat completely into the corner of the locks (9, 10, 11).

The word ‘comprising’ and forms of the word ‘comprising’ as used in this description and in the claims does not limit the invention claimed to exclude any variants or additions. Modifications and improvements to the invention will be readily apparent to those skilled in the art. Such modifications and improvements are intended to be within the scope of this invention.

1. A positioning bracket for a surgical procedure to assist in determining the correct placement and/or alignment of a surgical device for a patient, said positioning bracket comprising:
(a) a first arm mounted on a support frame;
(b) a rear positioning pad located on the first arm adjacent an end of the first arm for placement on the patient;
(c) a front positioning pad located on the first arm adjacent the other end of the first arm for placement on the patient; and
(d) a second arm mounted on the support frame, the second arm comprising a connector adjacent an end of the second arm to receive a surgical device;
wherein the support frame permits the connector to be positioned relative to the rear positioning pad and front positioning pad.

2. A positioning bracket according to claim 1 wherein the first arm is movable relative to the support frame.

3. A positioning bracket according to claim 1 wherein the first arm is movable along the support frame.

4. A positioning bracket according to claim 2 further comprising a lock to releasably fix the position of the first arm on the support frame.

5. A positioning bracket according to claim 1 wherein either or both the positioning pads is/are movable and/or adjustable relative to the first arm.

6. A positioning bracket according to claim 1 wherein the second arm is movable relative to the support frame.

7. A positioning bracket according to claim 1 wherein the second arm is movable along the support frame.

8. A positioning bracket according to claim 6 further comprising a lock to releasably fix the position of the second arm on the support frame.

9. A positioning bracket according to claim 1 wherein the connector is movable relative to the second arm.

10. A positioning bracket according to claim 1 wherein one end of the connector is slidingly engaged in an opening in the second arm.

11. A positioning bracket according to claim 1 wherein one end of the connector includes an opening to receive the surgical device.

12. A positioning bracket according to claim 1 wherein the support frame comprises a first support member and a second support member.

13. A positioning bracket according to claim 12 wherein the first arm is mounted on the first support member.

14. A positioning bracket according to claim 13 wherein the first arm is movable relative to the first support member.

15. A positioning bracket according to claim 13 wherein the first arm is movable along the first support member.

16. A positioning bracket according to claim 13 further comprising a lock to releasably fix the position of the first arm on the first support member.

17. A positioning bracket according to claim 12 wherein the second arm is mounted on the second support member.

18. A positioning bracket according to claim 17 wherein the second arm is movable relative to the second support member.

19. A positioning bracket according to claim 17 wherein the second arm is movable along the second support member.

20-23. (canceled)

24. A pelvic bracket for hip surgery to assist in determining the correct placement and/or alignment of an acetabulum cup for a patient, said pelvic bracket comprising:
(a) a first arm mounted on a support frame;
(b) a rear positioning pad located on the first arm adjacent an end of the first arm for placement on the sacrum of the patient;
(c) a front positioning pad located on the first arm adjacent the other end of the first arm and comprising three location sites, two of which are for placement on the patient's anterior superior iliac spine and the third is for placement on the patient's pubic tubercle; and
(d) a second arm mounted on the support frame, the second arm comprising a connector adjacent an end of the second arm to receive an acetabulum cup impactor;
wherein the support frame permits the connector to be positioned relative to the rear positioning pad and front positioning pad.
